(19) United States
(12) Patent Application Publication
(10) Pub. No.: US 2008/0234825 A1
(43) Pub. Date: Sep. 25, 2008

(54) Modular Lumbar Interbody Fixation Systems and Methods

(76) Inventor: James L. Chappuis, Marietta, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/852,370

(22) Filed: Sep. 10, 2007

Related U.S. Application Data
(60) Provisional application No. 60/895,244, filed on Mar. 16, 2007.

(51) Int. Cl. A61F 2/44; A61B 17/58; A61B 17/70

(52) U.S. Cl. 623/17.16; 606/246; 623/17.15

(57) ABSTRACT

Modular lumbar interbody fixation systems and methods are provided. A representative system includes: an anterior end opposite a posterior end; a pair of engaging plates adapted to fit between and engage the vertebral bodies to maintain a disc space between the vertebral bodies during use; and an alignment device positionable between the pair of engaging plates, the alignment device comprising at least one flexible strut extending between the pair of engaging plates during use.

### CROSS-REFERENCE TO RELATED APPLICATION

This application claims priority to copending U.S. provisional application entitled, “Modular Lumbar Interbody Fixation System,” having Ser. No. 60/895,244, filed on Mar. 16, 2007, which is entirely incorporated herein by reference.

### FIELD OF THE INVENTION

The present invention is generally related to promotion of spinal fusion between neighboring vertebrae.

### BACKGROUND OF THE INVENTION

The human spine is composed of a column of thirty-three bones, called vertebrae, and their adjoining structures. The twenty-four vertebrae nearest the head are separate bones and are capable of individual movement. These vertebrae are generally connected by anterior and posterior longitudinal ligaments and by discs of fibrocartilage, called intervertebral discs, positioned between opposing faces of adjacent vertebral bodies. The remaining nine vertebrae are fused to form the sacrum and the coccyx and are incapable of individual movement. The vertebral body and the dorsal vertebrae enclose an opening termed the vertebral foramen, through which the spinal cord, a column of nerve tissue which communicates nerve impulses between the brain and the rest of the body, and spinal nerve roots pass and are protected from damage.

Fusion of vertebral bodies may be required for any number of reasons. Most often, such fusion is necessitated when an intervertebral disc is damaged, degenerates, or otherwise becomes diseased, causing great discomfort by way of impinging on the spinal cord and/or nerve roots. When more conservative treatments and minimally invasive procedures have been exhausted, it may become necessary to surgically remove the damaged disk and fuse the associated vertebral bodies in order to restore the original spatial relationships, as well as desired stability.

After an intervertebral disc is removed, an implant device is typically inserted between neighboring vertebrae to maintain normal disc spacing and restore spinal stability, thereby facilitating an intervertebral fusion. An implant device typically contains a pair of engaging elements to engage the vertebrae. Prior to inserting the engaging elements, a vertebral drill is typically inserted within the surgical wound to drill into the cortical endplate and remove fibrous and nuclear material. A vertebral tap may then be used to cut threads into the ends of the neighboring vertebrae. The engaging elements are typically packed with bone graft to facilitate a spinal fusion.

Exemplary devices include those described in U.S. Pat. Nos. 6,045,579 and 6,080,193, which are incorporated herein by reference. These devices incorporate side struts, which are typically formed out of a material such as titanium and are relatively stiff. Because the struts are inflexible, the load distribution may result in accelerated subjacent degeneration of adjacent motion segments.

### SUMMARY OF THE INVENTION

Modular lumbar interbody fixation systems and methods are provided. An exemplary embodiment of such a system comprises: an anterior end opposite a posterior end; a pair of engaging plates adapted to fit between and engage the vertebral bodies to maintain a disc space between the vertebral bodies during use; and an alignment device positionable between the pair of engaging plates, the alignment device comprising at least one flexible strut extending between the pair of engaging plates during use.

Another exemplary embodiment of a system comprises: an anterior end opposite a posterior end; a first pair of engaging plates adapted to fit between and engage a first and a second vertebral body to maintain a disc space between the first and the second vertebral bodies during use, the first and the second vertebral bodies being adjacent in the spine; a first alignment device positionable between the first pair of engaging plates, the first alignment device comprising at least one strut extending between the first pair of engaging plates during use; a second pair of engaging plates adapted to fit between and engage a third and a fourth vertebral body to maintain a disc space between the third and the fourth vertebral bodies during use, the third and the fourth vertebral bodies being adjacent in the spine; and a second alignment device positionable between the second pair of engaging plates, the second alignment device comprising at least one flexible strut extending between the second pair of engaging plates during use; wherein the at least one flexible strut of the second alignment device is relatively more flexible than the at least one strut of the first alignment device.

An exemplary embodiment of a method comprises: removing an intervertebral disc between a first and a second vertebral bodies; inserting, between the first and the second vertebral bodies, a first spinal implant comprising: an anterior end opposite a posterior end, a pair of engaging plates, and an alignment device, the alignment device comprising at least one flexible strut.

Other systems, methods, features and/or advantages will be or may become apparent to one with skill in the art upon examination of the following drawings and detailed description. It is intended that all such additional systems, methods, features and/or advantages be included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 illustrates a side view of an embodiment of a modular lumbar interbody fixation system.

FIG. 2 illustrates a side view of another embodiment of a modular lumbar interbody fixation system.

### DETAILED DESCRIPTION OF THE PREFERRED EMBODIMENTS

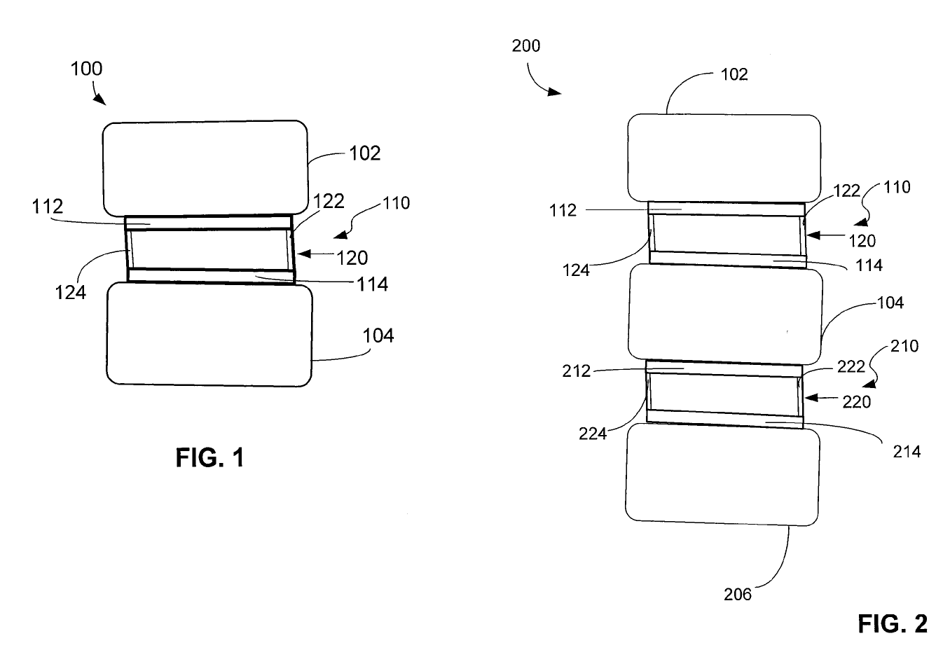

FIG. 1 illustrates an embodiment of a modular lumbar interbody fixation system 100. The modular lumbar interbody fixation system 100 comprises a spinal implant 110 configured to be inserted into the intervertebral space between adjacent vertebral bodies 102 and 104. Spinal implant 110 comprises engaging plates 112 and 114 and adjustment device 120. Spinal implant 110 is used to replace an intervertebral disc that has been removed for various reasons, including disease, degeneration, etc. Spinal implant 110 is adapted to maintain normal disc spacing, to restore spinal stability, and to facilitate a fusion between vertebral bodies 102 and 104.

Engaging plate 112 is configured to engage with vertebral body 102, and engaging plate 114 is configured to engage with vertebral body 104. Engaging plates 112 and 114 may be constructed of titanium, titanium alloy, ceramics, carbon composites, other metals, etc. Engaging plates 112 and 114 may be securely attached to vertebral bodies 102 and 104 respectively by way of, for example, cement between the respective surfaces, or pins and/or screws inserted through the engaging plate and into the vertebral body it engages. Engaging plates 112 and/or 114 may contain a plurality of openings disposed therein to allow bone development and growth through the engaging plates 112 and 114 and through spinal implant 110. The engaging surfaces of engaging plates 112 and 114 may be substantially planar to provide a relatively large contact area between the engaging plates and vertebral bodies 102 and 104. In this way, subsidence of the vertebral bodies 102 and 104 may be prevented because the force imparted to the vertebral bodies 102 and 104 from the spinal implant 110 is not concentrated across a relatively small area of the vertebral bodies. Alternatively, the engaging surfaces of engaging plates 112 and/or 114 may be non-planar. The engaging plates may contain a plurality of spikes or protrusions extending toward the vertebral bodies for enhancing an engagement between the vertebral body and the engaging plate. The protrusions may extend into vertebral bodies 102 and/or 104 to prevent spinal implant 110 from moving out of the intervertebral space.

Adjustment device 120 is configured to fit between engaging plates 112 and 114. Adjustment device 120 comprises two struts, 122 and 124, but adjustment device 120 may comprise one or more struts in other embodiments. As described herein, a “strut” is taken to mean any support member disposed between engaging plates 112 and 114 to separate engaging plates 112 and 114. Struts 122 and 124 may be attached to engaging plates 112 and 114 and/or attached directly to vertebral bodies 102 and 104. Unlike the relatively stiff struts described in the prior art, struts 122 and 124 are relatively flexible. By providing flexibility, better load distribution can be provided that may prevent accelerated subjacent degeneration of adjacent motion segments. Further, adjustment devices incorporating relatively flexible struts may provide a more stable anchoring of the spinal implant into vertebral bodies. In addition to being relatively flexible, which is a property dependent in part on the material, struts 122 and 124 may be configured and shaped to be deflectable (e.g., through the use of hinges) to cause stress to be applied to bone in the vicinity of spinal implant 110.

Struts 122 and 124 may have a predetermined height that defines the height of the spinal implant 110. Alternatively, struts 122 and 124 may each have an adjustable height. Engaging plates 112 and 114 may be configured to receive struts of various heights to allow the height of spinal implant 110 to be varied to fit the needs of the patient. In an embodiment, the struts 122 and 124 have differing heights to cause the height of spinal implant 110 to vary. In this manner, spinal implant 110 may be used to correct a lateral deviation in the spinal column as may occur in scoliosis. Struts 122 and 124 may contain a hinge pin to allow an upper member of the strut to pivot with respect to a lower member of the strut. In this manner, the struts may be pivoted such that the ends of the struts are properly aligned when a height difference exists between struts 122 and 124.

Spinal implant 110 may contain a retaining plate proximate the posterior end to provide a backing against which bone graft may be packed and to maintain bone graft between the engaging plates. The retaining plate may be substantially planar and may contain openings to allow bone ingrowth therethrough. A removable endcap may be positioned proximate the anterior end to contain bone graft within the fusion device and to prevent the migration of bone graft outside the engaging plates. The endcap may contain one or more openings for allowing bone ingrowth between a vertebral body and bone graft contained between the engaging plates. The endcap may be made of a plastic material, such as polyethylene, that tends to be non-irritating and non-abrasive to the surrounding tissues.

FIG. 2 illustrates an embodiment of a modular lumbar interbody fixation system 200. In modular lumbar interbody fixation system 200, three adjacent vertebral bodies are depicted, vertebral bodies 102, 104, and 206. Spinal implant 110, described above in connection with FIG. 1, is inserted in the intervertebral space between vertebral bodies 102 and 104. Additionally, spinal implant 210 is inserted in the intervertebral space between vertebral bodies 104 and 206. In other embodiments, spinal implant 210 may be inserted between vertebral bodies above or below vertebral bodies 102 and 104 and need not be adjacent to or inclusive of vertebral bodies 102 and 104.

Spinal implant 210 is similar to spinal implant 110, in that spinal implant 210 comprises engaging plates 212 and 214 and adjustment device 220. Engaging plates 212 and 214 are similar to engaging plates 112 and 114. Adjustment device 220, similar to adjustment device 120, comprises two struts, 222 and 224, but adjustment device 220 may comprise one or more struts in other embodiments. Struts 222 and 224 may be similar to struts 122 and 124, but may differ in stiffness. In particular, depending on the needs of the patient, struts 222 and 224 may be relatively more stiff than struts 122 and 124, or struts 222 and 224 may be relatively more flexible than struts 122 and 124. By using variable stiffness, subjacent degeneration can be reduced. Also, a form of anterior soft stabilization is provided.

In other embodiments, three or more spinal implant devices may be used, with the struts of each spinal implant device having varying degrees of flexibility. Clearly, in some applications, gradation from stiffer to more flexible devices can occur in both directions along the spine.

It should be emphasized that the above-described embodiments are merely possible examples of implementations. Many variations and modifications may be made to the above-described embodiments without departing substantially from the spirit and principles of the invention. All such modifications and variations are intended to be included herein within the scope of this disclosure and the present invention and protected by the following claims.

Therefore, having thus described the invention, at least the following is claimed:

1. A modular lumbar interbody fixation system for use with adjacent vertebral bodies comprising:
an anterior end opposite a posterior end;
a pair of engaging plates adapted to fit between and engage the vertebral bodies to maintain a disc space between the vertebral bodies during use; and
an alignment device positionable between the pair of engaging plates, the alignment device comprising at least one flexible strut extending between the pair of engaging plates during use.

2. The system of claim 1, wherein the at least one flexible strut comprises a hinge to allow an upper member of the at least one flexible strut to pivot with respect to a lower member of the at least one flexible strut during use such that the at least one flexible strut is able to flex and pivot.

3. The system of claim 1, wherein the alignment device is operable during use to adjust a height between the pair of engaging plates proximate the anterior end and simultaneously adjust a height between the pair of engaging plates proximate the posterior end.

4. The system of claim 1, wherein each of the engaging plates is substantially planar so as to inhibit subsidence of the vertebral bodies.

5. The system of claim 1, wherein at least one engaging plate comprises a plurality of openings to allow bone growth to occur through the at least one engaging plate.

6. The system of claim 5, further comprising bone graft adapted to be packed between the pair of engaging plates.

7. The system of claim 1, wherein the engaging plates comprise titanium.

8. The system of claim 1, wherein the at least one flexible strut is substantially deflectable to cause stress to be applied to bone in the vicinity of the implant.

9. The system of claim 1, further comprising protrusions extending from at least one of the engaging plates for enhancing an engagement between the vertebral body and the engaging plate, the protrusions being adapted to extend into the vertebral body.

10. A modular lumbar interbody fixation system for use with adjacent vertebral bodies comprising:
an anterior end opposite a posterior end;
a first pair of engaging plates adapted to fit between and engage a first and a second vertebral body to maintain a disc space between the first and the second vertebral bodies during use, the first and the second vertebral bodies being adjacent in the spine;
a first alignment device positionable between the first pair of engaging plates, the first alignment device comprising at least one strut extending between the first pair of engaging plates during use;
a second pair of engaging plates adapted to fit between and engage a third and a fourth vertebral body to maintain a disc space between the third and the fourth vertebral bodies during use, the third and the fourth vertebral bodies being adjacent in the spine; and
a second alignment device positionable between the second pair of engaging plates, the second alignment device comprising at least one flexible strut extending between the second pair of engaging plates during use;
wherein the at least one flexible strut of the second alignment device is relatively more flexible than the at least one strut of the first alignment device.

11. The system of claim 10, wherein the at least one flexible strut comprises a hinge to allow an upper member of the at least one flexible strut to pivot with respect to a lower member of the at least one flexible strut during use, and wherein the at least one strut comprises a hinge to allow an upper member of the at least one strut to pivot with respect to a lower member of the at least one strut during use.

12. The system of claim 10, wherein the each alignment device is operable during use to adjust a height between the respective pair of engaging plates proximate the anterior end and simultaneously adjust the height between the respective pair of engaging plates proximate the posterior end.

13. The system of claim 10, wherein each of the engaging plates is substantially planar so as to inhibit subsidence of the respective vertebral bodies.

14. The system of claim 10, wherein at least one engaging plate of at least one pair of engaging plates comprises a plurality of openings to allow bone growth to occur through the at least one engaging plate.

15. The system of claim 14, further comprising bone graft adapted to be packed between the at least one pair of engaging plates.

16. A method for promoting fusion between adjacent vertebral bodies comprising:
removing an intervertebral disc between a first and a second vertebral bodies;
inserting, between the first and the second vertebral bodies, a first spinal implant comprising:
an anterior end opposite a posterior end,
a pair of engaging plates, and
an alignment device, the alignment device comprising at least one flexible strut.

17. The method of claim 16, further comprising:
positioning the alignment device between the pair of engaging plates; and
engaging the first and the second vertebral bodies with the spinal implant.

18. The method of claim 17, further comprising:
inserting bone graft between the pair of engaging plates;
wherein at least one engaging plate of the pair of engaging plates comprises openings to allow bone growth to occur through the at least one engaging plate.

19. The method of claim 17, further comprising:
adjusting the alignment device such that the height between the engaging plates proximate the anterior end and the height between the engaging plates proximate the posterior end substantially maintains a natural curvature of the human spine when in use.

20. The method of claim 16, further comprising:
removing an intervertebral disc between a third and a fourth vertebral bodies, the second and the third vertebral bodies being identical or distinct; and
inserting, between the third and the fourth vertebral bodies, a second spinal implant comprising:
an anterior end opposite a posterior end,
a pair of engaging plates, and
an alignment device, the alignment device comprising at least one strut,
wherein the at least one strut is relatively less flexible than the at least one flexible strut of the first spinal implant.
